(19) United States
(12) Patent Application Publication
(10) Pub. No.: US 2022/0133287 A1
(43) Pub. Date: May 5, 2022

(54) SEALANT DELIVERY APPARATUS, AND SYSTEM AND METHOD FOR PREPARING SAME, FOR USE IN A LUNG PROCEDURE

(71) Applicant: C.R. Bard, Inc., Franklin Lakes, NJ (US)

(72) Inventors: Jordan P. Addison, Gilbert, AZ (US); Koltin K. Glaspie, Chandler, AZ (US); Heather A. Storm, Phoenix, AZ (US)

(73) Assignee: C.R. Bard, Inc., Franklin Lakes, NJ (US)

(21) Appl. No.: 17/431,928

(22) PCT Filed: Mar. 20, 2020
(86) PCT No.: PCT/US2020/023772

§ 371 Date: Aug. 18, 2021

Related U.S. Application Data
(60) Provisional application No. 62/822,490, filed on Mar. 22, 2019.

(51) Int. Cl. A61B 17/00; A61M 5/19; A61M 5/32; A61M 5/315

(52) U.S. Cl. CPC A61B 17/0057; A61M 5/19; A61M 5/3298; A61M 5/3286; A61M 2202/064; A61M 5/3294; A61B 2017/0065; A61B 2017/00809; A61B 2017/00477; A61M 5/31596

(57) ABSTRACT

A sealant delivery apparatus for use in a lung access procedure to aid in preventing pneumothorax includes a sealant applicator device and an injection needle assembly. The injection needle assembly has a hub and an elongate hollow stylet. The hub is configured for removable connection to the sealant applicator device. The elongate hollow stylet is configured to facilitate fluid communication with at least one output port of the sealant applicator device to receive a multi-component sealant. The elongate hollow stylet has a proximal portion, a distal portion having a closed distal end, and a plurality of side ports proximal to the closed distal end. The plurality of side ports radially extend from a lumen and through a side wall of the elongate hollow stylet in the distal portion. The plurality of side ports are in fluid communication with the at least one output port of the sealant applicator device.

### CROSS-REFERENCE TO RELATED APPLICATIONS

This application claims priority to U.S. provisional patent application Ser. No. 62/822,490 entitled “LUNG BIOPSY FLOWABLE SEALANT DELIVERY SYSTEM” filed Mar. 22, 2019, which is incorporated herein by reference.

### TECHNICAL FIELD

The present invention relates to a lung access procedure, such as a lung biopsy, and, more particularly, to a sealant delivery apparatus, and system and method for preparing the same, for use associated with a lung procedure to aid in preventing pneumothorax.

### BACKGROUND ART

Pneumothorax is a problematic complication of the lung biopsy procedure where air or fluid is allowed to pass into the pleural space as a result of the puncture of the parietal pleura and visceral pleura. Pneumothorax and, more so, pneumothorax requiring chest tube placement, are significant concerns for clinicians performing, and patients undergoing, percutaneous lung biopsies. The incidence of pneumothorax in patients undergoing percutaneous lung biopsy has been reported to be anywhere from 9-54%, with an average of around 15%. On average, 6.6% of all percutaneous lung biopsies result in pneumothorax requiring a chest tube to be placed, which results in an average hospital stay of 2.7 days.

Factors that increase the risk of pneumothorax include increased patient age, obstructive lung disease, increased depth of a lesion, multiple pleural passes, increased time that an access needle lies across the pleura, and traversal of a fissure. Pneumothorax may occur during or immediately after the procedure, which is why typically a CT scan of the region is performed following removal of the needle. Other, less common, complications of percutaneous lung biopsy include hemoptysis (coughing up blood), hemothorax (a type of pleural effusion in which blood accumulates in the pleural cavity), infection, and air embolism.

It has been suggested that approximately 30% of lung biopsies result in some form of pneumothorax that makes deploying a plug after the biopsy difficult or impossible. Current sealants on the market are deployed through a coaxial cannula after a biopsy is performed which may be before or after a pneumothorax forms.

What is needed in the art is a sealant delivery apparatus with the ability to deposit sealant components into the pleural space prior to a lung procedure, such as a biopsy, to aid in preventing pneumothorax, and a system and method for preparing the same.

### SUMMARY OF INVENTION

The present invention provides a sealant delivery apparatus with the ability to deposit sealant components into the pleural space prior to a lung procedure, such as a biopsy, to aid in preventing pneumothorax, and a system and method for preparing the same.

The invention, in one form, is directed to a sealant delivery apparatus for use in a lung access procedure to aid in preventing pneumothorax. The sealant delivery apparatus includes a sealant applicator device and an injection needle assembly. The sealant applicator device is configured to separately carry each of a first sealant component of a multi-component sealant and a second sealant component of the multi-component sealant. The sealant applicator device has at least one output port. The injection needle assembly has a hub and an elongate hollow stylet that extends distally from the hub. The hub is configured for removable connection to the sealant applicator device. The elongate hollow stylet is configured to facilitate fluid communication with the at least one output port of the sealant applicator device to receive the multi-component sealant. The elongate hollow stylet has a proximal portion and a distal portion. The distal portion has a closed distal end and a plurality of side ports proximal to the closed distal end. The elongate hollow stylet has a side wall that surrounds a lumen, wherein the plurality of side ports radially extend from the lumen and through the side wall of the elongate hollow stylet in the distal portion. The plurality of side ports are in fluid communication with the at least one output port of the sealant applicator device.

The invention, in another form, is directed to a system for preparing a sealant delivery apparatus for use in a lung access procedure. The system includes a first pair of syringes associated with the sealant delivery apparatus, a second pair of syringes, and a coupling mechanism. The first pair of syringes includes a first actuator, a first component chamber having a first component port, and a second component chamber having a second component port. The first actuator includes a first piston and a second piston. The first piston is positioned in the first component chamber and the second piston is positioned in the second component chamber. The first component chamber initially contains a first powder or solution component of a first sealant component of a multi-component sealant, and the second component chamber initially contains a second powder or solution component of the second sealant component of the multi-component sealant. The second pair of syringes includes a second actuator, a first component reservoir having a first transfer port, and a second component reservoir having a second transfer port. The second actuator includes a third piston and a fourth piston. The third piston is positioned in the first component reservoir and the fourth piston is positioned in the second component reservoir. The first component reservoir initially contains a first fluid component of the first sealant component of the multi-component sealant, and the second component reservoir initially contains a second fluid component of the second sealant component of the multi-component sealant. The coupling mechanism has a first coupling end and a second coupling end. The first coupling end is configured to releasably connect to the first pair of syringes and the second coupling end is configured to releasably connect to the second pair of syringes. The coupling mechanism has a first passage and a second passage. The first passage is configured to facilitate fluid communication between the first transfer port of the second pair of syringes and the first component port of the first pair of syringes, and the second passage is configured to facilitate fluid communication between the second transfer port of the second pair of syringes and the second component port of the first pair of syringes.

The invention, in another form, is directed to a method for preparing a sealant delivery apparatus for use in a lung access procedure. The method includes providing a first pair of syringes that includes a first actuator, a first component chamber having a first component port, and a second component chamber having a second component port, and wherein the first actuator includes a first piston and a second piston, the first piston being positioned in the first component chamber and the second piston being positioned in the second component chamber, the first component chamber containing a first powder or solution component of a first sealant component of a multi-component sealant, and the second component chamber containing a second powder or solution component of the second sealant component of the multi-component sealant; providing a second pair of syringes that includes a second actuator, a first component reservoir having a first transfer port, and a second component reservoir having a second transfer port, and wherein the second actuator includes a third piston and a fourth piston, the third piston being positioned in the first component reservoir and the fourth piston being positioned in the second component reservoir, the first component reservoir containing a first fluid component of the first sealant component of the multi-component sealant, and the second component reservoir containing a second fluid component of the second sealant component of the multi-component sealant; and providing a coupling mechanism having a first coupling end and a second coupling end, the first coupling end being releasably connectable to the first pair of syringes and the second coupling end being releasably connectable to the second pair of syringes, the coupling mechanism having a first passage and a second passage, wherein the first passage facilitates fluid communication between the first transfer port of the second pair of syringes and the first component port of the first pair of syringes, and wherein the second passage facilitates fluid communication between the second transfer port of the second pair of syringes and the second component port of the first pair of syringes.

An advantage of the present invention is that the invention improves upon typical solutions in that the multi-component sealant seals the pneumothorax region before the biopsy, rather than after the biopsy.

Another advantage of the present invention is that the invention improves over typical pneumothorax prevention devices in that, since the injection needle assembly is deployed at the beginning of the procedure rather than at the very end of a procedure, the flowable multi-component sealant, e.g., polymer, is able to be delivered and integrate into the spaces between tissues, whereas polymer plugs, for example, only occupy the space that they were cast and thus may result in a less effective seal.

Another advantage of the present invention is that the invention also requires no measuring of where the needle is in relation to the pleura beyond what physicians currently do. The solution of the present invention should be able to be seamlessly integrated into the lung access procedure by preparation of the flowable multi-component sealant, and dispensing the flowable multi-component sealant from the elongate hollow stylet of the injection needle assembly as the elongate hollow stylet is advanced across the pleura.

### BRIEF DESCRIPTION OF DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIG. 1 is a front view of the sealant delivery apparatus in accordance with an aspect of the present invention;

FIG. 2 is an exploded view of the sealant delivery apparatus of FIG. 1;

FIG. 3 is a side view of the sealant delivery device of the sealant delivery apparatus of FIG. 1, with the actuator retracted;

FIG. 4 is a section view of the sealant delivery device depicted in FIG. 3, taken along line 4-4 of FIG. 3, with the actuator retracted, and showing a first piston in a first component chamber proximal to a first sealant component of a multi-component sealant, and a second piston in a second component chamber proximal to a second sealant component of the multi-component sealant;

FIG. 5 is a perspective view of a distal portion of an elongate stylet of an injection needle assembly of the sealant delivery apparatus FIGS. 1 and 2;

FIG. 6 is a side view of the distal portion of the elongate stylet of the injection needle assembly of the sealant delivery apparatus FIGS. 1, 2, and 5;

FIG. 7 is a section view of the distal portion of the elongate stylet of the injection needle assembly of the sealant delivery apparatus FIGS. 1, 2, 5, and 6, taken along line 7-7 of FIG. 6;

FIG. 8 is a front view of a system for preparing the sealant delivery apparatus of FIGS. 1 and 2 for use in a lung access procedure, including the first pair of syringes and the coupling mechanism of the sealant delivery apparatus, and further including a second pair of syringes coupled to the first pair of syringes by the coupling mechanism;

FIG. 9 is an exploded view of the system of FIG. 8;

FIG. 10 is a side view of the system of FIG. 8, with the first actuator of the first pair of syringes retracted, and with the second actuator of a second pair of syringes retracted;

FIG. 11 is a section view of the system depicted in FIG. 10, taken along line 11-11 of FIG. 10, with the first actuator and the second actuator retracted, wherein a first component chamber of the first pair of syringes initially contains a powder or solution component of the first sealant component of the multi-component sealant, a second component chamber of the first pair of syringes initially contains a powder or solution component of the second sealant component of the multi-component sealant, a first component reservoir of the second pair of syringes initially contains a fluid component of the second sealant component of the multi-component sealant, and a second component reservoir of the second pair of syringes initially contains a fluid component of the second sealant component of the multi-component sealant; and

FIG. 12 is a flowchart of a method for preparing the sealant delivery apparatus of FIGS. 1 and 2, and more particularly the pair of syringes of the sealant delivery apparatus, for use in a lung access procedure.

Corresponding reference characters indicate corresponding parts throughout the several views. The exemplifications set out herein illustrate at least one embodiment of the invention, and such exemplifications are not to be construed as limiting the scope of the invention in any manner.

### DESCRIPTION OF EMBODIMENTS

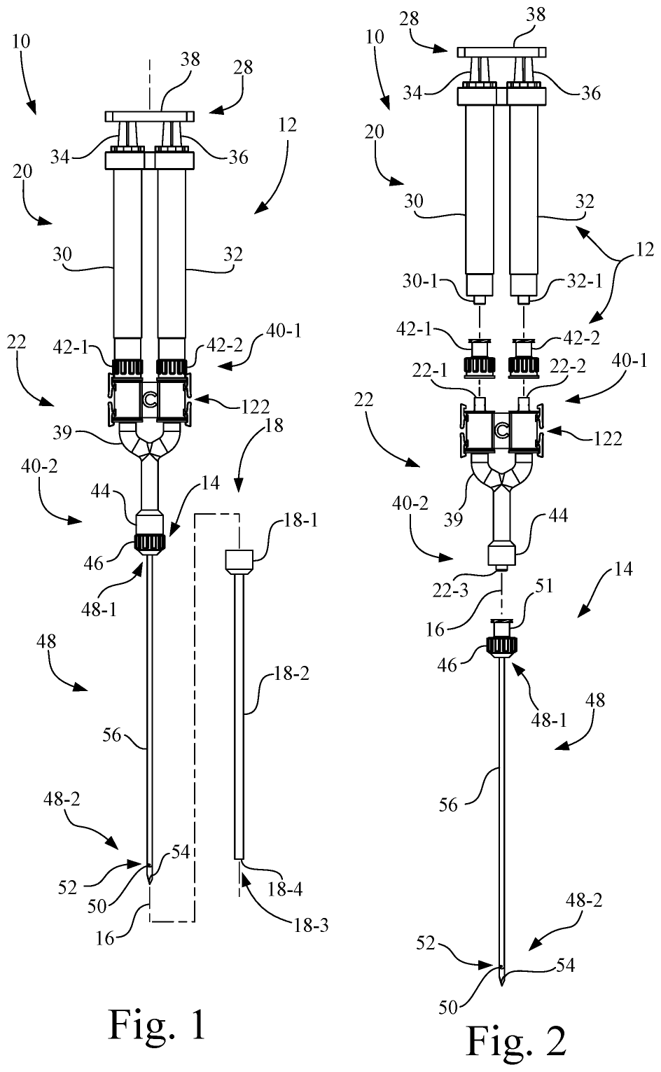

Referring now to the drawings, and more particularly to FIGS. 1 and 2, there is shown a sealant delivery apparatus 10 in accordance with an aspect of the present invention, for use in a lung access procedure to aid in preventing pneumothorax.

Sealant delivery apparatus 10 includes a sealant applicator device 12 and an injection needle assembly 14. Injection needle assembly 14 defines a longitudinal axis 16. When assembled, sealant delivery apparatus 10 has a configuration to facilitate delivery of a flowable multi-component sealant to an injection site, wherein the multi-component sealant may be injected while the needle portion of injection needle assembly 14 crosses the two layers of the pleura, i.e., the parietal and visceral pleura. Injection needle assembly 14 is configured to puncture a patient's tissue and create an access path, e.g., a proposed biopsy tract, in the tissue at least through the pleura of the patient, and thus allows for the two layers of the pleura to be sealed, e.g., prior to a lung biopsy, by the curing of the multi-component sealant at the delivery site so that air cannot leak between the two layers and cause a pneumothorax. It is noted that the multi-component sealant also may be deposited in other regions of the access path, such as in the subcutaneous tissue and/or lung parenchyma.

Optionally, sealant delivery apparatus 10 may be used in conjunction with an introducer cannula 18, wherein introducer cannula 18 may facilitate withdrawal of injection needle assembly 14 of sealant delivery apparatus 10 from the patient, while introducer cannula 18 may remain in place to maintain the access path in the patient's tissue to the site, such as for example, to receive and guide a second medical instrument, such as a biopsy device, to the site where the biopsy is to be performed.

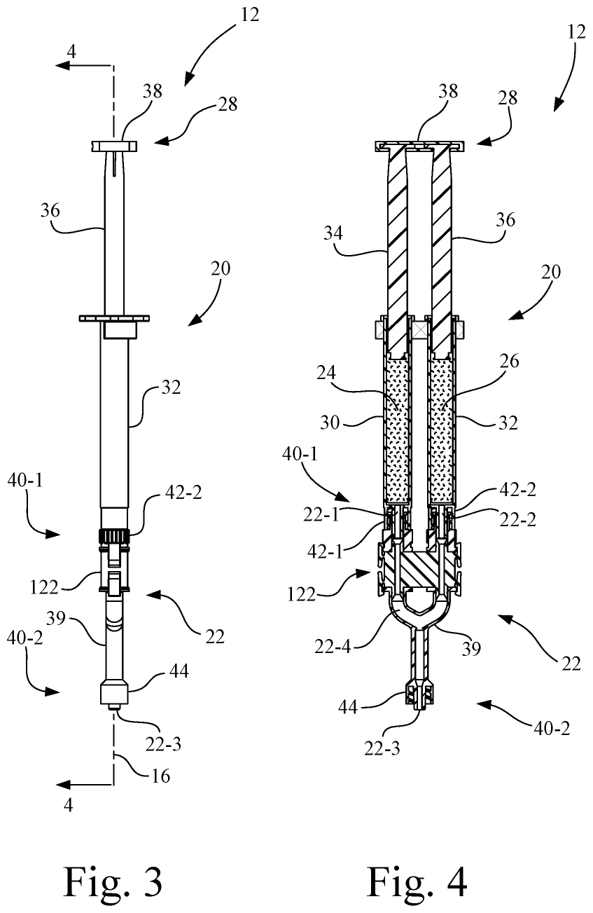

Referring also to FIGS. 3 and 4, sealant applicator device 12 includes a pair of syringes 20 and a manifold 22. Sealant applicator device 12 is configured to separately carry each of a first sealant component 24 of the multi-component sealant and a second sealant component 26 of the multi-component sealant. The first sealant component 24 may include, for example, at least two N-hydroxysuccinimide (NHS) ester groups, and the second sealant component 26 may include, for example, at least two amine groups. For example, the first sealant component 24 may be a solution containing polyethylene glycol (PEG) succinimidyl succinate and the second sealant component 26 may be a solution containing albumin and/or polyethylenimine (PEI). In the present embodiment, the first sealant component 24 and the second sealant component 26 are combined and mixed at manifold 22.

The pair of syringes 20 includes an actuator 28, a first component chamber 30, and a second component chamber 32. First component chamber 30 may be, for example, a cylindrical tube that is configured to carry the first sealant component 24 of the multi-component sealant. First component chamber 30 has a first component port 30-1. Second component chamber 32 also may be, for example, a cylindrical tube that is configured to carry the second sealant component 26 of the multi-component sealant. Second component chamber 32 has a second component port 32-1. First component chamber 30 and second component chamber 32 are arranged in a longitudinally parallel arrangement.

Actuator 28 includes a first piston 34, a second piston 36, and a handle 38. Handle 38 is in the form of a link member that perpendicularly extends between, and is connected to, each of first piston 34 and second piston 36 to facilitate simultaneous movement of first piston 34 and second piston 36 with the depression or retraction of handle 38. First piston 34 is in the form of a plunger that is positioned in first component chamber 30 proximal to the first sealant component 24, and second piston 36 is in the form of a plunger that is positioned in second component chamber 32 proximal to the second sealant component 26.

Referring to FIGS. 1-4, in the present embodiment, manifold 22 is removably connectable at a first coupling end 40-1 to the pair of syringes 20 of sealant applicator device 12 by a pair of connecters 42-1, 42-2, and is removably connectable at a second end 40-2 to injection needle assembly 14 by a connector 44. More particularly, as best shown in FIG. 4, manifold 22 includes a Y-connector 39 releasably connected to a coupling mechanism 122, wherein coupling mechanism 122 serves as a detachable extension of the two-port portion of Y-connector 39. Thus, Y-connector 39 (and more particularly, coupling mechanism 122) is removably connectable at first coupling end 40-1 to the pair of syringes 20 of sealant applicator device 12 by the pair of connecters 42-1, 42-2, and Y-connector 39 is removably connectable at a second end 40-2 to injection needle assembly 14 by a connector 44.

The pair of connecters 42-1, 42-2 may form a threaded, or alternatively a snap-fit, connection with the pair of syringes 20 of sealant applicator device 12. Likewise, connector 44 may form a threaded, or alternatively a snap-fit, connection with injection needle assembly 14.

Those skilled in the art will recognize that the pair of connecters 42-1, 42-2 may be included as a part of the pair of syringes 20, and releasably connectable to manifold 22. Alternatively, the pair of connecters 42-1, 42-2 may be included as a part of manifold 22, and releasably connectable to the pair of syringes 20. Further, alternatively, the pair of connecters 42-1, 42-2 may be included as separate parts, each of which is separately connectable to each of the pair of syringes 20 and the manifold 22.

Manifold 22 has a first input port 22-1, a second input port 22-2, and at least one output port, which in the present embodiment is a single output port, referenced hereinafter as output port 22-3. As best shown in FIG. 4, in the present embodiment, coupling mechanism 122 of manifold 22 has individual passages in fluid communication with an internal Y-passageway 22-4 of Y-connector 39. In the present embodiment, coupling mechanism 122 of manifold 22 directly includes first input port 22-1 and second input port 22-2, and Y-connector 39 of manifold 22 directly includes output port 22-3. Thus, by virtue of Y-connector 39, the at least one output port, e.g., output port 22-3, is in fluid communication with each of first input port 22-1 and second input port 22-2. Also, when manifold 22 is connected to the pair of syringes 20 of sealant applicator device 12, then first input port 22-1 is in fluid communication with first component chamber 30 and second input port 22-2 is in fluid communication with second component chamber 32.

Referring to FIGS. 1 and 2, injection needle assembly 14 includes a hub 46 and an elongate hollow stylet 48 that extends distally from hub 46. Elongate hollow stylet 48 has a proximal portion 48-1 and a distal portion 48-2. Hub 46 is fixedly attached, e.g., through overmolding, adhesive and/or pressed fit, to proximal portion 48-1 of elongate hollow stylet 48. Hub 46 is configured for removable connection to sealant applicator device 12. More particularly, hub 46 of injection needle assembly 14 may include a connector 51 (see FIG. 2) that may form a threaded, or alternatively a snap-fit, connection with connector 44 at second end 40-2 of manifold 40 of sealant applicator device 12.

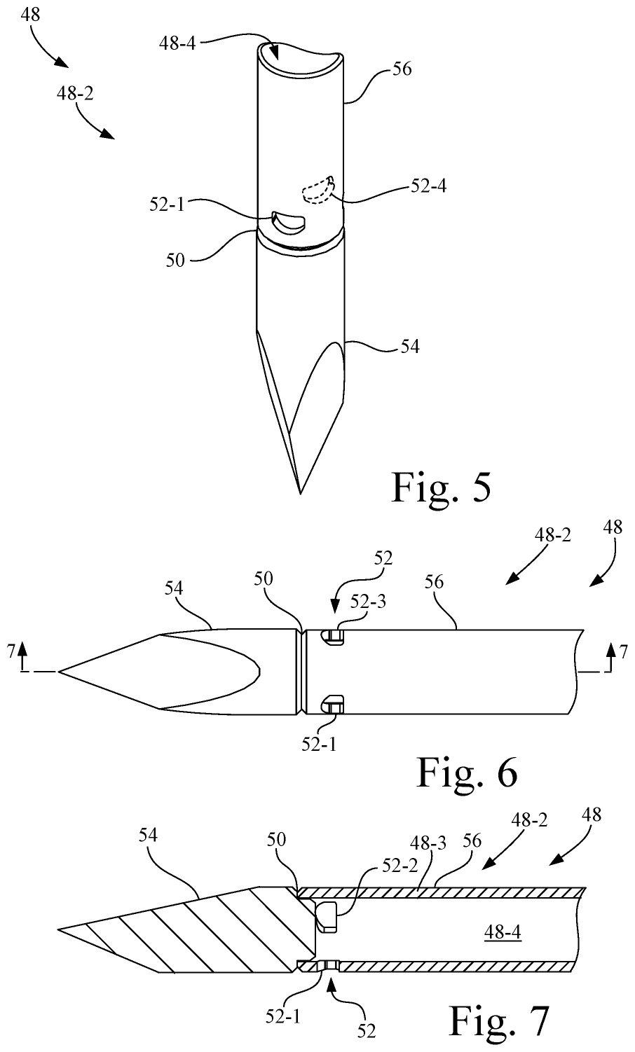

Elongate hollow stylet 48 of injection needle assembly 14 is configured to facilitate fluid communication with the at least one output port, e.g., output port 22-3 of manifold 22 of sealant applicator device 12 so as to receive the multi-component sealant from sealant applicator device 12. Referring also to FIGS. 5-7, distal portion 48-2 has a closed distal end 50 and a plurality of side ports 52 (e.g., at least three) proximal to closed distal end 50. Closed distal end 50 of elongate hollow stylet 48 may be, for example, a closed stylet needle tip 54.

Elongate hollow stylet 48, may be constructed, for example, by an elongate cannula 56 being fixedly connected to the closed stylet needle tip 54, with the plurality of side ports 52 being located in elongate cannula 56 immediately proximal to closed stylet needle tip 54. More particularly, elongate cannula 56 of elongate hollow stylet 48 defines a side wall 48-3 that surrounds a lumen 48-4 of elongate hollow stylet 48, wherein the plurality of side ports 52 radially extend from lumen 48-4 and through side wall 48-3 of elongate hollow stylet 48 in distal portion 48-2. Closed stylet needle tip 54 is defined, at least in part, by closed distal end 50 of elongate hollow stylet 48, wherein closed stylet needle tip 54 is attached, e.g., welded, press fit, or with adhesive, to elongate cannula 56 to distally close lumen 48-4 of elongate cannula 56 of elongate hollow stylet 48. Stated differently, closed stylet needle tip 54 terminates a distal extent of lumen 48-4 of elongate hollow stylet 48. When sealant delivery apparatus 10 is assembled, the plurality of side ports 52 are in fluid communication with the at least one output port, e.g., output port 22-3, of manifold 22 of sealant applicator device 12 by way of lumen 48-4 of elongate hollow stylet 48.

In the present embodiment, the plurality of side ports 52 in distal portion 48-2 of elongate hollow stylet 48 includes a side port 52-1, a side port 52-2, and a side port 52-3, which are located in distal portion 48-2 and arranged, e.g., in a ring pattern, around a perimeter of elongate hollow stylet 48, such as in 120 degree increments. In one application, for example, it is preferred that the plurality of side ports 52 include at least three side ports (e.g., 3 to 7 side ports) to have the at least three side ports near, but proximal to, closed stylet needle tip 54 such that the flowable multi-component sealant may be delivered 360 degrees around elongate hollow stylet 48, so as to seal around the perforation/close to the perforation created by closed stylet needle tip 54. As another example, it is contemplated that in some applications it may be desirable to have the plurality of side ports 52 configured as two, or two pairs, of diametrically opposed side ports.

Optionally, as shown by phantom lines in FIG. 5, it is further contemplated that the plurality of side ports 52 may include at least two longitudinally spaced side ports, such as for example, a side port 52-4 longitudinally spaced (e.g. 1 to 3 millimeters) proximal to side port 52-1. For example, the plurality of side ports 52 may include two rings of three side ports arranged around a perimeter of elongate hollow stylet 48, wherein the two rings of three side ports are longitudinally spaced in distal portion 48-2 of elongate hollow stylet 48.

Referring again to FIG. 1, injection needle assembly 14 may be used in conjunction with the introducer cannula 18 (also sometimes referred to in the art as a coaxial introducer needle) to allow elongate hollow stylet 48 to be removed from introducer cannula 18, while maintaining access to the procedure site with the coaxial introducer needle. In other words, sealant delivery apparatus 10 may be removed from introducer cannula 18 and replaced with a variety of other instruments, such as a biopsy device or another stylet. In the present embodiment, introducer cannula 18 has a coaxial hub 18-1, a coaxial cannula 18-2, a cannula lumen 18-3 and a distal annular rim 18-4. Cannula lumen 18-3, e.g., is configured, e.g., having a cylindrical shape, to receive elongate hollow stylet 48 of injection needle assembly 14. When elongate hollow stylet 48 of injection needle assembly 14 is fully inserted into cannula lumen 18-3 of introducer cannula 18 (e.g., distal movement of elongate hollow stylet 48 is stopped by contact of hub 46 of injection needle assembly 14 with coaxial hub 18-1 of introducer cannula 18, the plurality of side ports 52 of elongate hollow stylet 48 are located distal to the distal annular rim 18-4 of introducer cannula 18.

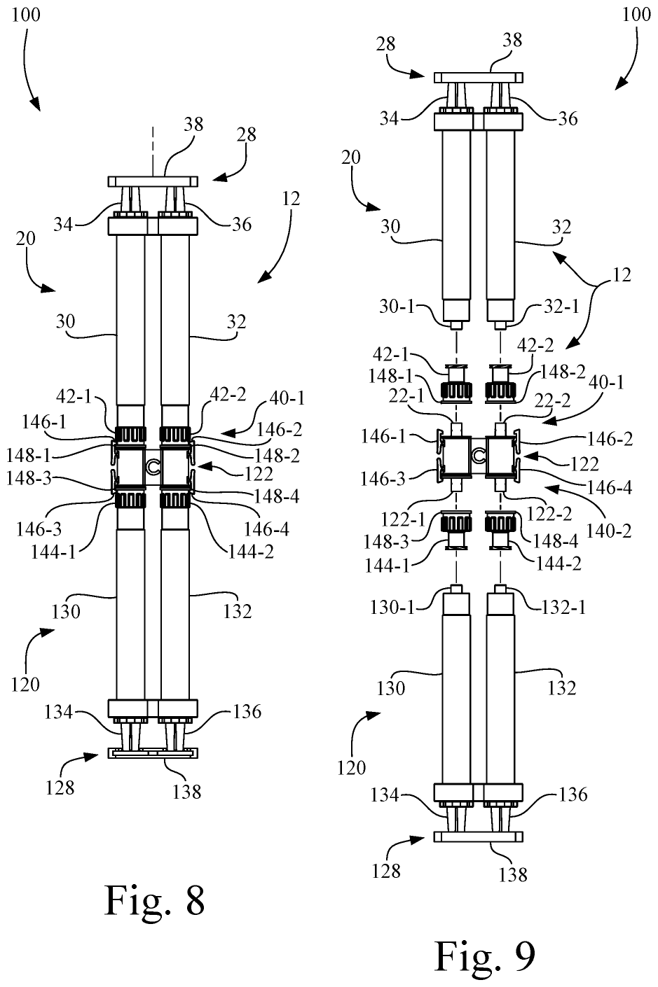

Referring to FIGS. 8-11, there is show a system 100 for preparing sealant delivery apparatus 10 for use in a lung access procedure. More particularly, system 100 is used to mix various chemical agents, which when combined will result in the first sealant component 24 and the second sealant component 26 of the multi-component sealant depicted in FIG. 4. System 100 generally includes the first pair of syringes 20, a second pair of syringes 120, and coupling mechanism 122.

As described above, the first pair of syringes 20 is configured having first component chamber 30, second component chamber 32, and the first actuator 28 having first piston 34 and second piston 36. However, initially (e.g., as a deliverable from the manufacturer), first component chamber 30 does not yet contain the prepared first sealant component 24 and second component chamber 32 does not yet contain the prepared second sealant component 26, as depicted in FIG. 4. Rather, referring to FIG. 11, initially, first component chamber 30 initially contains a powder or solution component 24-1 of the first sealant component 24 of the multi-component sealant, and second component chamber 32 initially contains a powder or solution component 26-1 of the second sealant component 26 of the multi-component sealant. As used herein, each solution forming component 24-1 and/or component 26-1 is a solute and solvent combination, and may include, for example, a suspension or hydrogel.

Referring to FIGS. 8-11, the second pair of syringes 120 includes a second actuator 128, a first component reservoir 130 having a first transfer port 130-1, and a second component reservoir 132 having a second transfer port 132-1.

Second actuator 128 includes a third piston 134, a fourth piston 136, and a handle 138. Handle 138 is in the form of a link member that perpendicularly extends between, and is connected to, each of third piston 134 and fourth piston 136 to facilitate simultaneous movement of third piston 134 and fourth piston 136 with the depression or retraction of handle 138. Third piston 134 is in the form of a plunger that is positioned in first component reservoir 130 and fourth piston 136 is in the form of a plunger that is positioned in second component reservoir 132. First component reservoir 130 initially contains a fluid component 24-2 of the first sealant component 24 of the multi-component sealant, and second component reservoir 132 initially contains a fluid component 26-2 of the second sealant component 26 of the multi-component sealant. As used herein, fluid component 24-2 and/or fluid component 26-2 may be, or include, water or some other liquid.

Coupling mechanism 122 has a first coupling end 40-1 and a second coupling end 140-2. First coupling end 40-1 is configured to releasably connect to the first pair of syringes 20, and second coupling end 140-2 is configured to releasably connect to the second pair of syringes 120. Referring particularly to FIGS. 9 and 11, coupling mechanism 122 includes first input port 22-1 and second input port 22-2 at first coupling end 40-1, and includes a port 122-1 and a port 122-2 at second coupling end 140-2. Coupling mechanism 122 further includes a first passage 150-1 in fluid communication with, and extending between, port 22-1 and port 122-1. Likewise, coupling mechanism 122 includes a second passage 150-2 in fluid communication with, and extending between, port 22-2 and port 122-2.

The pair of connecters 42-1, 42-2 may form a threaded, or alternatively a snap-fit, connection between the first pair of syringes 20 and first coupling end 40-1 of coupling mechanism 122. Likewise, a pair of connecters 144-1, 144-2 may form a threaded, or alternatively a snap-fit, connection between the second pair of syringes 120 and second coupling end 140-2 of coupling mechanism 122.

The pair of connecters 42-1, 42-2 may be included as a part of the first pair of syringes 20, and releasably connectable to coupling mechanism 122. Alternatively, the pair of connecters 42-1, 42-2 may be included as a part of coupling mechanism 122, and releasably connectable to the first pair of syringes 20. As a further alternative, the pair of connecters 42-1, 42-2 may be included as separate parts, each of which is separately connectable to each of the pair of syringes 20 and coupling mechanism 122.

Likewise, the pair of connecters 144-1, 144-2 may be included as a part of the second pair of syringes 120, and releasably connectable to coupling mechanism 122. Alternatively, the pair of connecters 144-1, 144-2 may be included as a part of coupling mechanism 122, and releasably connectable to the second pair of syringes 120. As a further alternative, the pair of connecters 144-1, 144-2 may be included as separate parts, each of which is separately connectable to each of the second pair of syringes 120 and coupling mechanism 122.

Referring to FIGS. 8-11, first coupling end 40-1 of coupling mechanism 122 includes a first snap latch 146-1 and a second snap latch 146-2. First component chamber 30 of the first pair of syringes 20 (but more particularly connector 42-1) includes a first snap catch 148-1 configured to releasably engage first snap latch 146-1 of coupling mechanism 122. Similarly, second component chamber 32 of the first pair of syringes 20 (but more particularly connector 42-2) includes a second snap catch 148-2 configured to releasably engage second snap latch 146-2 of coupling mechanism 122.

Second coupling end 140-2 of coupling mechanism 122 includes a third snap latch 146-3 and a fourth snap latch 146-4. First component reservoir 130 of the second pair of syringes 120 (but more particularly connector 144-1) includes a third snap catch 148-3 configured to releasably engage third snap latch 146-3 of coupling mechanism 122. Similarly, second component reservoir 132 of the second pair of syringes 120 (but more particularly connector 144-2) has a fourth snap catch 148-4 configured to releasably engage fourth snap latch 146-4 of coupling mechanism 122.

Referring to FIGS. 9 and 11, first passage 150-1 of coupling mechanism 122 is configured to facilitate fluid communication between first transfer port 130-1 of the second pair of syringes 120 and first component port 30-1 of the first pair of syringes 20, and second passage 150-2 of coupling mechanism 122 is configured to facilitate fluid communication between second transfer port 132-1 of the second pair of syringes 120 and second component port 32-1 of the first pair of syringes 20.

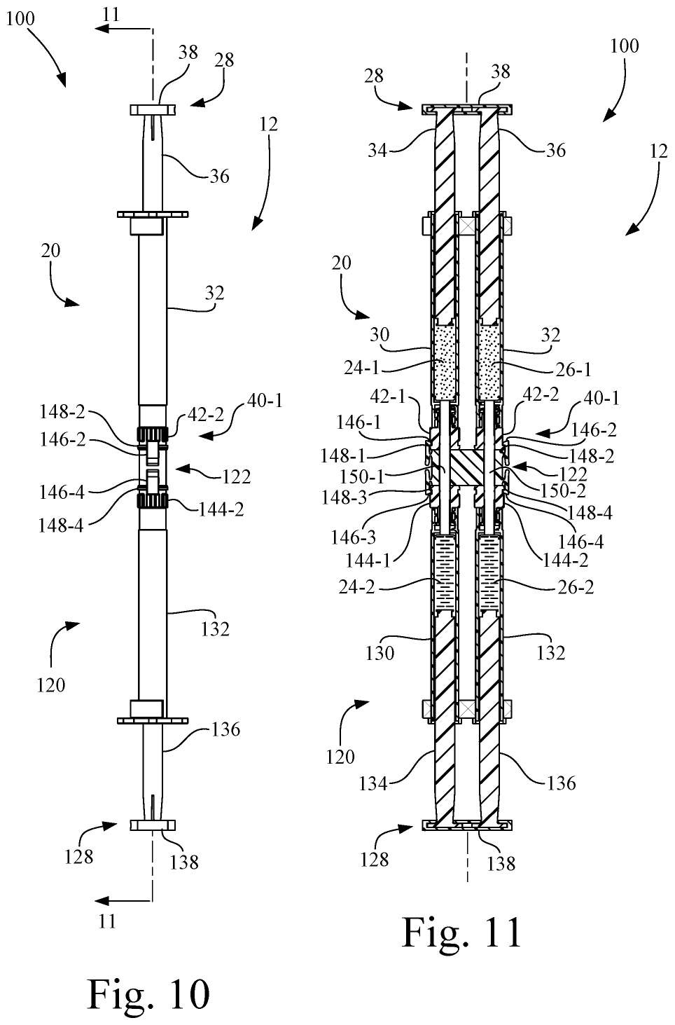

Referring to FIGS. 10 and 11, first actuator 28 of the first pair of syringes 20 is configured to simultaneously move first piston 34 and second piston 36, and second actuator 128 of the second pair of syringes 120 is configured to simultaneously move third piston 134 and fourth piston 136.

Accordingly, by alternatingly operating, e.g., depressing, second actuator 128 and first actuator 28, the second pair of syringes 120 and the first pair of syringes 20 are configured to alternatingly transfer any contents of first component reservoir 130 and second component reservoir 132 to first component chamber 30 and second component chamber 32, respectively, and to transfer any contents of first component chamber 30 and second component chamber 32 to first component reservoir 130 and second component reservoir 132, respectively. For example, the first pair of syringes 20 and the second pair of syringes 120 may be configured to simultaneously: (A) hydrate powder or solution component 24-1 with fluid component 24-2 to form the first sealant component 24 of the multi-component sealant, wherein when fully hydrated, the first sealant component 24 of the multi-component sealant resides in first component chamber 30 of the first pair of syringes 20 (see also FIG. 4), and (B) hydrate powder or solution component 26-1 with fluid component 26-2 to form the second sealant component 26 of the multi-component sealant, wherein when fully hydrated, the second sealant component 26 of the multi-component sealant resides in second component chamber 32 of the first pair of syringes 20, as depicted in FIG. 4.

For example, in a first depression of second actuator 128, third piston 134 and fourth piston 136 simultaneously transfer fluid component 24-2 of the first sealant component 24 through first passage 150-1 into first component chamber 30 carrying powder or solution component 24-1 of the first sealant component 24 and transfers fluid component 26-2 of the second sealant component 26 through second passage 150-2 into second component chamber 32 carrying powder or solution component 26-1 of the second sealant component 26. Thereafter, in a first depression of first actuator 28, first piston 34 and second piston 36 simultaneously transfer the contents of first component chamber 30 and the contents of second component chamber 32 of the first pair of syringes 20 into first component reservoir 130 and second component reservoir 132, respectively, of the second pair of syringes 120. Then, in a second depression of second actuator 128, third piston 134 and fourth piston 136 simultaneously transfer the contents of first component reservoir 130 and second component reservoir 132 of the second pair of syringes 120 into first component chamber 30 and second component chamber 32, respectively, of the first pair of syringes 20. This process may be repeated, as necessary, to ensure the adequate hydration of powder or solution component 24-1 with fluid component 24-2 to form the first sealant component 24 of the multi-component sealant, and to ensure the adequate hydration of powder or solution component 26-1 with fluid component 26-2 to form the second sealant component 26 of the multi-component sealant.

At the conclusion of hydration/mixing, first sealant component 24 resides in first component chamber 30 and second sealant component 26 resides in second component chamber 32 of the first pair of syringes 20. Coupling mechanism 122 may then be decoupled from the first pair of syringes 20, then manifold 22 may be coupled to first pair of syringes 20, and then injection needle assembly 14 may be coupled to the first pair of syringes 20 via manifold 22 to form the sealant delivery apparatus 10, as described above with respect to FIGS. 1-7.

Alternatively, at the conclusion of hydration/mixing, coupling mechanism 122 may then be decoupled from the second pair of syringes 120, then manifold 22 may be formed by coupling Y-connector 39 to coupling mechanism 122, and then injection needle assembly 14 may be coupled to the first pair of syringes 20 via manifold 22 to form the sealant delivery apparatus 10, as described above with respect to FIGS. 1-7.

It is noted that a kit may be formed by a combination of the sealant delivery apparatus 10 (assembled or disassembled) of FIG. 1 and/or FIG. 2 with the pair of connecters 144-1, 144-2 and the second pair of syringes 120 of FIG. 9.

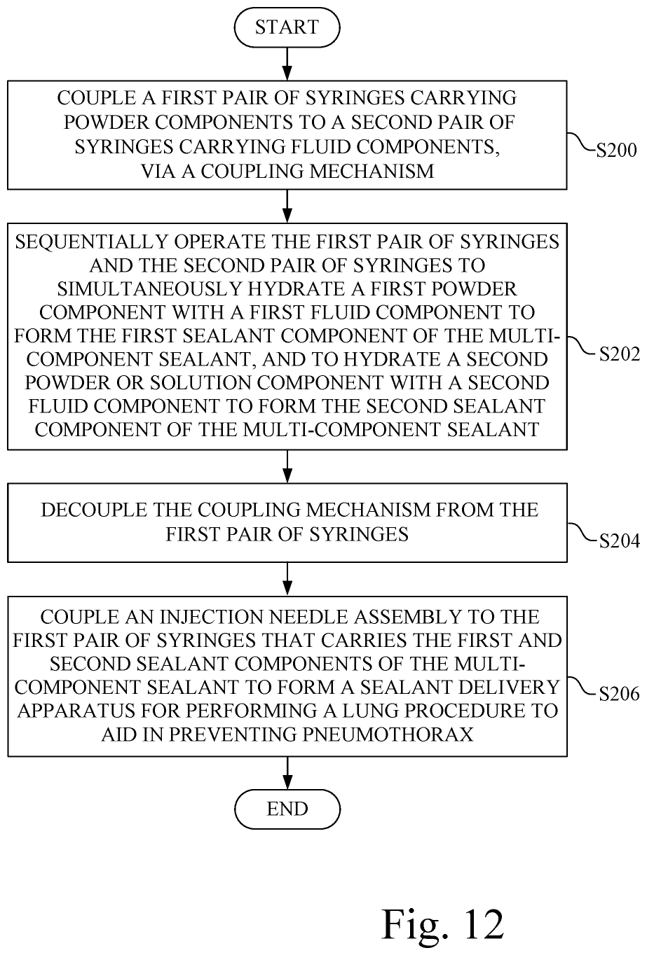

FIG. 12 is a flowchart of a method for preparing sealant delivery apparatus 10, and more particularly the pair of syringes 20 of sealant delivery apparatus 10, for use in a lung access procedure, wherein the following method steps may be sequentially performed.

At step S200, the first pair of syringes 20 are coupled to the second pair of syringes 120 via coupling mechanism 122.

At step S202, the first pair of syringes 20 and the second pair of syringes 120 are sequentially (i.e., alternatingly) operated to simultaneously hydrate powder or solution component 24-1 with fluid component 24-2 to form the first sealant component 24 of the multi-component sealant, and to hydrate powder or solution component 26-1 with fluid component 26-2 to form the second sealant component 26 of the multi-component sealant.

The step of sequentially operating the first pair of syringes 20 and the second pair of syringes 120 may be performed by alternatingly depressing second actuator 128 of the second pair of syringes 120 and first actuator 28 of the first pair of syringes 20 to alternatingly transfer any contents of first component reservoir 130 and second component reservoir 132 to first component chamber 30 and second component chamber 32, respectively, and transfer any contents of first component chamber 30 and second component chamber 32 to first component reservoir 130 and second component reservoir 132

When fully hydrated, the first sealant component 24 of the multi-component sealant resides in first component chamber 30 of the first pair of syringes 20, and the second sealant component 26 of the multi-component sealant resides in second component chamber 32 of the first pair of syringes 20. To accomplish this, a desired amount of the first sealant component 24 and the second sealant component 26 are moved into the first pair of syringes 20 by depressing the second actuator 128 of the second pair of syringes 120 until the desired amount moves into the first pair of syringes 20.

At step S204, coupling mechanism 122 may be decoupled from the first pair of syringes 20, and manifold 22 may be coupled to the first pair of syringes 20. Alternatively, coupling mechanism 122 may be decoupled from the second pair of syringes 120, and manifold 22 may be formed by coupling Y-connector 39 to coupling mechanism 122.

At step S206, injection needle assembly 14 is coupled to the first pair of syringes 20, e.g., via manifold 22 (see FIG. 1), to form the sealant delivery apparatus 10, as described above with respect to FIGS. 1-7, which is now ready for performing a lung procedure to aid in preventing pneumothorax.

In a method of use of the prepared sealant delivery apparatus 10 described above at steps S200-S206, elongate hollow stylet 48 is inserted into the patient to create an access path, and actuator 28 of the pair of syringes 20 may be depressed as the access path is being created so as to deliver the multi-component sealant from the plurality of side ports 52 into the access path at the subcutaneous tissue, pleura layers, pleura space, and/or lung parenchyma as the access path is being created.

The following items also relate to the invention:

The invention, in one form, is related to a sealant delivery apparatus for a lung access procedure, in particular for prevention of pneumothorax. The sealant delivery apparatus includes a sealant applicator device and an injection needle assembly. The sealant applicator device may be configured to separately carry each of a first sealant component of a multi-component sealant and a second sealant component of the multi-component sealant. The sealant applicator device has at least one output port. The injection needle assembly has a hub and an elongate hollow stylet that extends distally from the hub. The hub may be configured for removable connection to the sealant applicator device. The elongate hollow stylet may be configured to facilitate (provide) fluid communication with the at least one output port of the sealant applicator device to receive the multi-component sealant. The elongate hollow stylet has a proximal portion and a distal portion. The distal portion has a closed distal end and a plurality of side ports proximal to (in the vicinity of) the closed distal end. The elongate hollow stylet has a side wall that surrounds a lumen, wherein the plurality of side ports radially extend from the lumen and through the side wall of the elongate hollow stylet in the distal portion. The plurality of side ports are in fluid communication with the at least one output port of the sealant applicator device.

In some embodiments, the plurality of side ports in the distal portion of the elongate hollow stylet comprises at least three side ports in the distal portion arranged around a perimeter of the elongate hollow stylet.

In some embodiments, the plurality of side ports in the distal portion of the elongate hollow stylet includes at least two longitudinally spaced side ports.

In some embodiments, the closed distal end of the elongate hollow stylet may be a closed needle tip that terminates a distal extent of the lumen.

In some embodiments, the elongate hollow stylet may comprise an elongate cannula that defines the side wall and the lumen, and a stylet needle tip may be defined by the closed distal end, wherein the stylet needle tip may be attached to the elongate cannula to distally close the lumen of the elongate cannula.

In any of the embodiments, the sealant applicator device may include a pair of syringes having an actuator, a first component chamber that may be configured to carry the first sealant component of the multi-component sealant, and a second component chamber that may be configured to carry the second sealant component of the multi-component sealant. The actuator may include a first piston and a second piston. The first piston may be positioned in the first component chamber proximal to the first sealant component, and the second piston may be positioned in the second component chamber proximal to the second sealant component. A manifold may include a first input port, a second input port, and the at least one output port, wherein the at least one output port is in fluid communication with each of the first input port and the second input port. The first input port is in fluid communication with the first component chamber and the second input port is in fluid communication with the second component chamber.

In some embodiments, the manifold may be a Y-connector that has an internal Y-passageway that includes the first input port, the second input port, and the at least one output port configured as a single output port. The single output port may be in fluid communication with the plurality of side ports in the distal end of the elongate hollow stylet having the closed distal end.

Optionally, the apparatus may comprise an introducer cannula having a cannula lumen and a distal annular rim. The cannula lumen may be configured to receive the elongate hollow stylet of the injection needle assembly, wherein when the elongate hollow stylet of the injection needle assembly is fully inserted into the cannula lumen of the introducer cannula, the plurality of side ports are located distal to the distal annular rim of the introducer cannula.

The invention may also relate to a lung biopsy apparatus comprising the sealant delivery apparatus as described above.

The invention, in another form, is related to a system for preparing a sealant delivery apparatus for a lung access procedure. The system includes a first pair of syringes associated with the sealant delivery apparatus, a second pair of syringes, and a coupling mechanism. The first pair of syringes may include a first actuator, a first component chamber having a first component port, and a second component chamber having a second component port. The first actuator may include a first piston and a second piston. The first piston is positioned in the first component chamber and the second piston is positioned in the second component chamber. The first component chamber initially contains a first powder or solution component of a first sealant component of a multi-component sealant, and the second component chamber initially contains a second powder or solution component of the second sealant component of the multi-component sealant. The second pair of syringes may include a second actuator, a first component reservoir having a first transfer port, and a second component reservoir having a second transfer port. The second actuator may include a third piston and a fourth piston. The third piston is positioned in the first component reservoir and the fourth piston is positioned in the second component reservoir. The first component reservoir initially contains a first fluid component of the first sealant component of the multi-component sealant, and the second component reservoir initially contains a second fluid component of the second sealant component of the multi-component sealant. The coupling mechanism has a first coupling end and a second coupling end. The first coupling end may be configured to releasably connect to the first pair of syringes and the second coupling end may be configured to releasably connect to the second pair of syringes. The coupling mechanism may have a first passage and a second passage. The first passage may be configured to facilitate fluid communication between the first transfer port of the second pair of syringes and the first component port of the first pair of syringes, and the second passage may be configured to facilitate fluid communication between the second transfer port of the second pair of syringes and the second component port of the first pair of syringes.

In some system embodiments, the first actuator of the first pair of syringes may be configured to simultaneously move the first piston and the second piston, and the second actuator of the second pair of syringes may be configured to simultaneously move the third piston and the fourth piston.

In any of the system embodiments, the second pair of syringes may be configured such that a first depression of the second actuator simultaneously transfers the first fluid component of the first sealant component into the first component chamber carrying the first powder or solution component of the first sealant component and transfers the second fluid component of the second sealant component into the second component chamber carrying the second powder or solution component of the second sealant component.

In any of the system embodiments, the first pair of syringes and the second pair of syringes may be configured to alternatingly transfer any contents of the first component reservoir and the second component reservoir to the first component chamber and the second component chamber, respectively, and to transfer any contents of the first component chamber and the second component chamber to the first component reservoir and the second component reservoir, respectively, by alternatingly depressing the second actuator and the first actuator.

In any of the system embodiments, the first pair of syringes and the second pair of syringes may be configured to hydrate the first powder or solution component with the first fluid component to form the first sealant component of the multi-component sealant, wherein when fully hydrated, the first sealant component of the multi-component sealant resides in the first component chamber of the first pair of syringes. Also, the first pair of syringes and the second pair of syringes may be configured to hydrate the second powder or solution component with the second fluid component to form the second sealant component of the multi-component sealant, wherein when fully hydrated, the second sealant component of the multi-component sealant resides in the second component chamber of the first pair of syringes.

Optionally, the first coupling end of the coupling mechanism may include a first snap latch and a second snap latch, and the second coupling end of the coupling mechanism may include a third snap latch and a fourth snap latch. Also, a first snap catch may be associated with the first component chamber of the first pair of syringes, wherein the first snap catch is configured to releasably engage the first snap latch of the coupling mechanism. A second snap catch may be associated with the second component chamber of the first pair of syringes, wherein the second snap catch is configured to releasably engage the second snap latch of the coupling mechanism. A third snap catch may be associated with the first component reservoir of the second pair of syringes, wherein the third snap catch is configured to releasably engage the third snap latch of the coupling mechanism. A fourth snap catch may be associated with the second component reservoir of the second pair of syringes, wherein the fourth snap catch is configured to releasably engage the fourth snap latch of the coupling mechanism.

In any of the system embodiments, the system may optionally include an injection needle assembly having a hub and an elongate hollow stylet that extends distally from the hub. The hub may be configured for removable connection to the sealant applicator device. The elongate hollow stylet may be configured to facilitate fluid communication with the first component port and the second component port of the sealant applicator device to receive the multi-component sealant. The elongate hollow stylet has a proximal portion and a distal portion. The distal portion may have a closed distal end and a plurality of side ports proximal to the closed distal end. The elongate hollow stylet may have a side wall that surrounds a lumen, wherein the plurality of side ports radially extend from the lumen and through the side wall of the elongate hollow stylet in the distal portion. The plurality of side ports are configured for fluid communication with the first component port and the second component port of the sealant applicator device.

The invention may also relate to a lung biopsy system comprising the system as described above.

The invention may also relate to a combination comprising the sealant delivery apparatus as described above and the system for preparing a sealant delivery apparatus as described above.

The invention, in another form, is related to a method for preparing a sealant delivery apparatus for use in a lung access procedure. The method may comprise providing a first pair of syringes that includes a first actuator, a first component chamber having a first component port, and a second component chamber having a second component port, and wherein the first actuator includes a first piston and a second piston, the first piston may be positioned in the first component chamber and the second piston may be positioned in the second component chamber, the first component chamber containing a first powder or solution component of a first sealant component of a multi-component sealant, and the second component chamber containing a second powder or solution component of the second sealant component of the multi-component sealant; providing a second pair of syringes that includes a second actuator, a first component reservoir having a first transfer port, and a second component reservoir having a second transfer port, and wherein the second actuator includes a third piston and a fourth piston, the third piston may be positioned in the first component reservoir and the fourth piston may be positioned in the second component reservoir, the first component reservoir containing a first fluid component of the first sealant component of the multi-component sealant, and the second component reservoir containing a second fluid component of the second sealant component of the multi-component sealant; and providing a coupling mechanism having a first coupling end and a second coupling end, the first coupling end may be releasably connectable to the first pair of syringes and the second coupling end may be releasably connectable to the second pair of syringes, the coupling mechanism having a first passage and a second passage, wherein the first passage facilitates fluid communication between the first transfer port of the second pair of syringes and the first component port of the first pair of syringes, and wherein the second passage facilitates fluid communication between the second transfer port of the second pair of syringes and the second component port of the first pair of syringes.

In some embodiments, the method may include the sequential steps of: connecting the first pair of syringes to the second pair of syringes via the coupling mechanism; moving the first actuator of the first pair of syringes to simultaneously move the first piston and the second piston; and moving the second actuator of the second pair of syringes to simultaneously move the third piston and the fourth piston.

In any of the method embodiments, the method may include connecting the first pair of syringes to the second pair of syringes via the coupling mechanism; and depressing the second actuator of the second pair of syringes to both transfer the first fluid component of the first sealant component from the first component reservoir into the first component chamber carrying the first powder or solution component of the first sealant component, and to simultaneously transfer the second fluid component of the second sealant component from the second component reservoir into the second component chamber carrying the second powder or solution component of the second sealant component.

In any of the method embodiments, the method may include connecting the first pair of syringes to the second pair of syringes via the coupling mechanism; sequentially operating the first pair of syringes and the second pair of syringes to hydrate the first powder or solution component with the first fluid component to form the first sealant component of the multi-component sealant, wherein when fully hydrated, the first sealant component of the multi-component sealant resides in the first component chamber of the first pair of syringes; and wherein simultaneous with the act of sequentially operating the first pair of syringes and the second pair of syringes, the first pair of syringes and the second pair of syringes operate to hydrate the second powder or solution component with the second fluid component to form the second sealant component of the multi-component sealant, wherein when fully hydrated, the second sealant component of the multi-component sealant resides in the second component chamber of the first pair of syringes.

In some of the method embodiments, the method may include alternatingly depressing the second actuator of the second pair of syringes and the first actuator of the first pair of syringes to alternatingly transfer any contents of the first component reservoir and the second component reservoir to the first component chamber and the second component chamber, respectively, and transfer any contents of the first component chamber and the second component chamber to the first component reservoir and the second component reservoir.

In some of the method embodiments, the method may include decoupling the coupling mechanism from the first pair of syringes; and coupling an injection needle assembly to the first pair of syringes.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

1. A sealant delivery apparatus for use in a lung access procedure to aid in preventing pneumothorax, comprising:
a sealant applicator device configured to separately carry each of a first sealant component of a multi-component sealant and a second sealant component of the multi-component sealant, the sealant applicator device having at least one output port; and
an injection needle assembly having a hub and an elongate hollow stylet that extends distally from the hub, the hub configured for removable connection to the sealant applicator device, the elongate hollow stylet configured to facilitate fluid communication with the at least one output port of the sealant applicator device to receive the multi-component sealant,
the elongate hollow stylet having a proximal portion and a distal portion, the distal portion having a closed distal end and a plurality of side ports proximal to the closed distal end, the elongate hollow stylet having a side wall that surrounds a lumen, wherein the plurality of side ports radially extend from the lumen and through the side wall of the elongate hollow stylet in the distal portion, the plurality of side ports being in fluid communication with the at least one output port of the sealant applicator device, and wherein the plurality of side ports in the distal portion of the elongate hollow stylet includes at least two longitudinally spaced side ports.

2. The apparatus according to claim 1, wherein the plurality of side ports in the distal portion of the elongate hollow stylet comprises at least three side ports in the distal portion arranged around a perimeter of the elongate hollow stylet.

3. The apparatus according to claim 1, wherein the plurality of side ports in the distal portion of the elongate hollow stylet includes a first side port longitudinally spaced 1 to 3 millimeters proximal to a second side port.

4. The apparatus according to claim 1, wherein the closed distal end of the elongate hollow stylet is a closed needle tip that terminates a distal extent of the lumen.

5. The apparatus according to claim 1, wherein the elongate hollow stylet comprises:
an elongate cannula that defines the side wall and the lumen; and
a stylet needle tip defined by the closed distal end, wherein the stylet needle tip is attached to the elongate cannula to distally close the lumen of the elongate cannula.

6. The apparatus according to claim 1, wherein the sealant applicator device includes:
a pair of syringes having an actuator, a first component chamber configured to carry the first sealant component of the multi-component sealant, and a second component chamber configured to carry the second sealant component of the multi-component sealant, and wherein the actuator includes a first piston and a second piston, the first piston being positioned in the first component chamber proximal to the first sealant component, and the second piston being positioned in the second component chamber proximal to the second sealant component; and
a manifold that includes a first input port, a second input port, and the at least one output port, wherein the at least one output port is in fluid communication with each of the first input port and the second input port, and wherein the first input port is in fluid communication with the first component chamber and the second input port is in fluid communication with the second component chamber.

7. The apparatus according to claim 6, wherein the manifold is a Y-connector that has an internal Y-passageway that includes the first input port, the second input port, and the at least one output port configured as a single output port, the single output port being in fluid communication with the plurality of side ports in the distal end of the elongate hollow stylet having the closed distal end.

8. The apparatus according to claim 1, further comprising an introducer cannula having a cannula lumen and a distal annular rim, the cannula lumen configured to receive the elongate hollow stylet of the injection needle assembly, wherein when the elongate hollow stylet of the injection needle assembly is fully inserted into the cannula lumen of the introducer cannula, the plurality of side ports are located distal to the distal annular rim of the introducer cannula.

9. A system for preparing a sealant delivery apparatus for use in a lung access procedure, comprising:
the sealant delivery apparatus including a first pair of syringes, wherein the first pair of syringes includes a first actuator, a first component chamber having a first component port, and a second component chamber having a second component port, and wherein the first actuator includes a first piston and a second piston, the first piston being positioned in the first component chamber and the second piston being positioned in the second component chamber, the first component chamber initially containing a first powder or solution component of a first sealant component of a multi-component sealant, and the second component chamber initially containing a second powder or solution component of the second sealant component of the multi-component sealant;
a second pair of syringes that includes a second actuator, a first component reservoir having a first transfer port, and a second component reservoir having a second transfer port, and wherein the second actuator includes a third piston and a fourth piston, the third piston being positioned in the first component reservoir and the fourth piston being positioned in the second component reservoir, the first component reservoir initially containing a first fluid component of the first sealant component of the multi-component sealant, and the second component reservoir initially containing a second fluid component of the second sealant component of the multi-component sealant; and
a coupling mechanism having a first coupling end and a second coupling end, the first coupling end configured to releasably connect to the first pair of syringes and the second coupling end configured to releasably connect to the second pair of syringes, the coupling mechanism having a first passage and a second passage, wherein the first passage is configured to facilitate fluid communication between the first transfer port of the second pair of syringes and the first component port of the first pair of syringes, and wherein the second passage is configured to facilitate fluid communication between the second transfer port of the second pair of syringes and the second component port of the first pair of syringes, and
wherein the first pair of syringes and the second pair of syringes are configured to alternatingly transfer any contents of the first component reservoir and the second component reservoir to the first component chamber and the second component chamber, respectively, and to transfer any contents of the first component chamber and the second component chamber to the first component reservoir and the second component reservoir, respectively, by alternatingly depressing the second actuator and the first actuator.

10. The system according to claim 9, wherein the first actuator of the first pair of syringes is configured to simultaneously move the first piston and the second piston, and wherein the second actuator of the second pair of syringes is configured to simultaneously move the third piston and the fourth piston.

11. The system according to claim 9, wherein the second pair of syringes is configured such that a first depression of the second actuator simultaneously transfers the first fluid component of the first sealant component into the first component chamber carrying the first powder or solution component of the first sealant component and transfers the second fluid component of the second sealant component into the second component chamber carrying the second powder or solution component of the second sealant component.

12. The system according to claim 9, wherein the second actuator and the first actuator are alternatingly depressed until the respective dry component is fully hydrated by the respective fluid component.

13. The system according to claim 9, wherein:
the first pair of syringes and the second pair of syringes are configured to hydrate the first powder or solution component with the first fluid component to form the first sealant component of the multi-component sealant, wherein when fully hydrated, the first sealant component of the multi-component sealant resides in the first component chamber of the first pair of syringes; and
the first pair of syringes and the second pair of syringes are configured to hydrate the second powder or solution component with the second fluid component to form the second sealant component of the multi-component sealant, wherein when fully hydrated, the second sealant component of the multi-component sealant resides in the second component chamber of the first pair of syringes.

14. The system according to claim 9, wherein the first coupling end of the coupling mechanism includes a first snap latch and a second snap latch, and the second coupling end of the coupling mechanism includes a third snap latch and a fourth snap latch, and further comprising:
a first snap catch associated with the first component chamber of the first pair of syringes, the first snap catch configured to releasably engage the first snap latch of the coupling mechanism;
a second snap catch associated with the second component chamber of the first pair of syringes, the second snap catch configured to releasably engage the second snap latch of the coupling mechanism;
a third snap catch associated with the first component reservoir of the second pair of syringes, the third snap catch configured to releasably engage the third snap latch of the coupling mechanism; and
a fourth snap catch associated with the second component reservoir of the second pair of syringes, the fourth snap catch configured to releasably engage the fourth snap latch of the coupling mechanism.

15. The system according to claim 9, further comprising an injection needle assembly having a hub and an elongate hollow stylet that extends distally from the hub, the hub configured for removable connection to the sealant applicator device, the elongate hollow stylet configured to facilitate fluid communication with the first component port and the second component port of the sealant applicator device to receive the multi-component sealant, the elongate hollow stylet having a proximal portion and a distal portion, the distal portion having a closed distal end and a plurality of side ports proximal to the closed distal end, the elongate hollow stylet having a side wall that surrounds a lumen, wherein the plurality of side ports radially extend from the lumen and through the side wall of the elongate hollow stylet in the distal portion, the plurality of side ports configured for fluid communication with the first component port and the second component port of the sealant applicator device.

16. A method for preparing a sealant delivery apparatus for use in a lung access procedure, comprising:
providing a first pair of syringes that includes a first actuator, a first component chamber having a first component port, and a second component chamber having a second component port, and wherein the first actuator includes a first piston and a second piston, the first piston being positioned in the first component chamber and the second piston being positioned in the second component chamber, the first component chamber containing a first powder or solution component of a first sealant component of a multi-component sealant, and the second component chamber containing a second powder or solution component of the second sealant component of the multi-component sealant;
providing a second pair of syringes that includes a second actuator, a first component reservoir having a first transfer port, and a second component reservoir having a second transfer port, and wherein the second actuator includes a third piston and a fourth piston, the third piston being positioned in the first component reservoir and the fourth piston being positioned in the second component reservoir, the first component reservoir containing a first fluid component of the first sealant component of the multi-component sealant, and the second component reservoir containing a second fluid component of the second sealant component of the multi-component sealant;
providing a coupling mechanism having a first coupling end and a second coupling end, the first coupling end being releasably connectable to the first pair of syringes and the second coupling end being releasably connectable to the second pair of syringes, the coupling mechanism having a first passage and a second passage, wherein the first passage facilitates fluid communication between the first transfer port of the second pair of syringes and the first component port of the first pair of syringes, and wherein the second passage facilitates fluid communication between the second transfer port of the second pair of syringes and the second component port of the first pair of syringes; and
alternatingly depressing the second actuator of the second pair of syringes and the first actuator of the first pair of syringes to alternatingly transfer any contents of the first component reservoir and the second component reservoir to the first component chamber and the second component chamber, respectively, and transfer any contents of the first component chamber and the second component chamber to the first component reservoir and the second component reservoir.

17. The method according to claim 16, comprising the sequential steps of:
connecting the first pair of syringes to the second pair of syringes via the coupling mechanism;
moving the first actuator of the first pair of syringes to simultaneously move the first piston and the second piston; and
moving the second actuator of the second pair of syringes to simultaneously move the third piston and the fourth piston.

18. The method according to claim 16, comprising:
connecting the first pair of syringes to the second pair of syringes via the coupling mechanism;
depressing the second actuator of the second pair of syringes to both transfer the first fluid component of the first sealant component from the first component reservoir into the first component chamber carrying the first powder or solution component of the first sealant component, and to simultaneously transfer the second fluid component of the second sealant component from the second component reservoir into the second component chamber carrying the second powder or solution component of the second sealant component.

19. The method according to claim 16, comprising:
connecting the first pair of syringes to the second pair of syringes via the coupling mechanism;
sequentially operating the first pair of syringes and the second pair of syringes to hydrate the first powder or solution component with the first fluid component to form the first sealant component of the multi-component sealant, wherein when fully hydrated, the first sealant component of the multi-component sealant resides in the first component chamber of the first pair of syringes; and
wherein simultaneous with the act of sequentially operating the first pair of syringes and the second pair of syringes, the first pair of syringes and the second pair of syringes operate to hydrate the second powder or solution component with the second fluid component to form the second sealant component of the multi-component sealant, wherein when fully hydrated, the second sealant component of the multi-component sealant resides in the second component chamber of the first pair of syringes.

20. The method according to claim 17, wherein the act of alternatingly depressing the second actuator of the second pair of syringes and the first actuator of the first pair of syringes continues until the respective powder or solution component is fully hydrated by the respective fluid component.

21. (canceled)
